(19) United States
(12) Patent Application Publication
(10) Pub. No.: US 2025/0102486 A1
(43) Pub. Date: Mar. 27, 2025

(54) Method for assessing the stability and quality of dairy products and plant-based milk alternatives over the storage period

(71) Applicant: DMK Deutsches Milchkontor GmbH, Zeven (DE)

(72) Inventors: Katja BODE, Hollenstedt (DE); Maren Christina SCHULZ, Wilstedt (DE); Ralf ZINK, Bad Zwischenahn (DE)

(21) Appl. No.: 18/895,845

(22) Filed: Sep. 25, 2024

(30) Foreign Application Priority Data
Sep. 25, 2023 (EP) 23199337.9

(51) Int. Cl. G01N 33/04

(52) U.S. Cl. CPC G01N 33/04

(57) ABSTRACT

A method is proposed to assess the stability and quality of dairy products, primary and main milk-based products including cheeses and plant-based milk alternatives over the storage period by aging dairy products, primary and main milk-based products including cheeses or plant-based milk alternatives under defined conditions, taking regular samples and analyzing them using different parameters. The data sets are analyzed using Principal Component Analysis (PCA) and the Partial Least Square (PLS) calibration method. The calculated ages are compared with the actual sample age and provide a calibration line that can be used to determine the age of unknown samples.

### AREA OF INVENTION

The present invention relates to the field of food technology and concerns a method for the early assessment of the stability and quality, in particular the age, of dairy products, products based primarily and principally on milk, including cheeses or their plant-based milk alternatives, by collecting defined quality and composition data and statistically evaluating them.

### TECHNOLOGICAL BACKGROUND

One of the greatest requirements in the food industry is not only to manufacture and market its products in perfect quality, but also to ensure this quality and techno-functionality for a sufficiently long period of time. In addition to the microbiological shelf life, which can be ensured by suitable processes such as heating, aseptic filling and packaging, as well as preservation, chemical or physical changes to the product during the storage period must also be avoided, as these can at best only lead to changes in flavor, but at worst can spoil the food and have a negative impact on its techno-functionality. One of the most important factors influencing the quality of food is the permeation of substances, in particular oxygen, through the packaging material, which can affect the color, taste of the food, techno-functionality or all three factors as a result of oxidation. The packaging used must therefore guarantee optimum product and functionality protection and meet high product-specific requirements in terms of both material selection and packaging design. In particular, perfect quality and techno-functionality of the product must be ensured within the declared minimum shelf life.

In addition to material testing and theoretical calculation models, standard qualification tests, such as product storage tests in real time, are also available to determine the quality, functionality and, derived from this, the minimum shelf life. The packaging materials to be tested are filled and stored under controlled conditions for the specified shelf life. However, this means that a sufficiently reliable statement about the suitability of a packaging design for an existing product or, conversely, a new product development for an existing type of packaging can only be made after the intended minimum shelf life has expired. This very long test period of several months in some cases directly highlights the main disadvantage of this test method. Due to the ever-increasing variety of products, increasing transportation routes and ever-shorter product cycles, combined with high consumer expectations regarding product quality and safety, there is a need to reduce the required development time in the competition between product suppliers.

### RELATED STATE OF THE ART

Relevant methods are already known from the prior art which can be used to indirectly and imprecisely determine the shelf life of food in a package. For example, EP 1626275 B1 (WILD) proposes a method for assessing the shelf life of a product in a packaging material, which comprises the following steps:
(a) placing a sealed packaging means (4) filled with product in a pressurized chamber (1),
(b) storing the packaging material (4) in the pressurized chamber (1) for a period of time t1 at a positive pressure p1 and a specific temperature T1 in a test gas atmosphere, whereby a defined quantity Q of test gas permeates into the packaging material,
(c) storing the packaging material (4) for a specific period of time t2 under the influence of heat and/or light, and
(d) analytical and/or sensory examination of the filling material.

A disadvantage of the method is the high cost of the apparatus; in addition, the functional principle-simulation of the amount of oxygen introduced into the filling material and acceleration of the reaction by increased pressure-ultimately only permits a very rough estimate, which is often unsuitable for a qualitative statement, especially since it is known that for many polymer/gas systems the solubility and diffusion coefficient of the permeating substance in the polymer (e.g. of a plastic bottle) can vary from one polymer to another. (e.g., a plastic bottle) become dependent on concentration and pressure if the investigation is carried out below the glass temperature Tg of the polymers and/or the critical temperature of the gas [MÜLLER, K. “Oxygen permeability of plastic bottles and caps” Dissertation TU München (2003)]

A completely different method of visually indicating the shelf life of a packaged foodstuff to the consumer is the subject of EP 2378354 B1, EP 2581785 B1 and WO 2009 056591 A1 (UNI MÜNSTER). These relate to a sensor device, specifically an electrochemical processor, which is short-circuited when the packaging is opened. An aluminum oxide layer is formed, the progression of which is proportional to the shelf life of the product. An increase in temperature, for example, accelerates the formation of oxide and thus naturally also reflects the decreasing degree of freshness of the product. However, this solution makes it necessary to equip each individual package with a separate sensor, which is also technically complex and can often show unstable results with multiple or single openings.

EP 3875953 B1 (DMK) describes an accelerated storage test for determining the quality and shelf life of foodstuffs, in which a first sample is stored at a temperature T1 and a similar second sample is stored at a significantly higher temperature T2, whereby the samples are sensory assessed and compared with each other via a calibration curve. As this sensory evaluation of products always contains a subjective component, a method based exclusively on objective parameters would be more advantageous.

### OBJECT OF THE INVENTION

Therefore, the object of the present invention has been to provide a test method which, by recourse to a calibration diagram, allows certain properties, such as the age or quality of an unknown milk product, primary and main milk-based products including cheeses or their milk alternative products, to be indicated quickly and reliably.

### DESCRIPTION OF THE INVENTION

The invention relates to a method of assessing the stability and quality of milk products, primary or main milk-based products including cheese and plant-based milk alternatives over storage time, comprising or consisting of the following steps:
(a) providing a first set of samples (P1.0, P2.0 . . . Pn.0) of a dairy product, a primary or main milk-based product including cheese, or a plant-based dairy alternative;
(b) ageing the samples from step (a) over defined periods of time to obtain an nth set of samples (P1.1, P1.2 . . . P2.1, P2.2 . . . Pn.x);
(c) creating a set of test parameters;
(d) applying the specified test parameters to each of the individual samples of the first and nth sample set, generating a data set;
(e) checking the correlation of the data in the data set using Principal Component Analysis (PCA) to obtain a coordinate system (score plot) and determining the largest variance of the data as the first component and the second largest variance of the data as the second component;
(f) performing a Partial Least Square Analysis (PLS) on the data;
(g) generating a partial least square (PLS) calibration model to obtain a PLS response plot (calibration line), wherein the calculated response for each data point is plotted against the corresponding measured response of each data point; and
(h) evaluating an external sample with respect to the specified evaluation criterion using the calibration grades.

Surprisingly, it was found that in particular the age and thus the quality of a dairy product, products on a primary and main basis of milk including cheeses or vegetable milk alternatives can be easily and reliably determined on the basis of selected chemical and physical data, which are usually collected in any case as part of quality control.

The method according to the invention is divided into four sections:
selection of suitable analysis parameters;
data generation;
checking the correlation of the data by PCA; and
calculation of the product age using PLS.
   Dairy Products, Products Based Primarily and Mainly on Milk, Including Cheeses or their Plant-Based Milk Alternatives

The selection of dairy products, products on a primary and main basis of milk including cheeses or plant-based milk alternatives, to which the method according to the invention can be applied, is in itself not critical. Typically, the products are selected from the group formed by whole milk, skimmed milk, UHT milk, milk powders, cream, whey, curd, cheese and yoghurt, but also products on a primary and main basis of milk, such as numerous desserts. Also included are their plant-based milk alternatives such as plant-based drinks, spreads, cream alternatives, yoghurt alternatives and cheese alternatives.

### Sample Sets

Before the data set can be created, the samples must first be prepared. The first sample set comprises the samples at time t=0, i.e., the fresh samples, which are referred to below as P1, P2, P3 to Pn. The first set of samples usually comprises 3 to 10 and preferably 4 to 7 samples, whereby these are packaged in containers that typically have a volume of 10 ml to 100 L.

To prepare the second and each further (“n-th”) set of samples, the original samples are stored (“aged”) under defined conditions, preferably darkened at temperatures of 4 to 20° C. The storage period can cover a period of 1 to 25 weeks, but longer storage periods of 4 to 22 weeks are preferable. During this time, further samples are taken at regular intervals, typically every 1-2 weeks. After the first ageing period of 2 weeks, the original sample 2 becomes the aged sample 2 after the 1st ageing period with the designation P2.1. A sample P4.11 would then be the designation for the original sample 4, which has undergone 11 ageing periods, i.e., is 22 weeks old.

### Test Parameters

An essential feature of the method according to the invention is the selection of the test parameters on the basis of which the samples are to be evaluated. The following are preferably considered for this purpose:
dissolved oxygen quantity;
pH value;
instability index (Lumisizer);
brightness color value white/black (L*);
color coordinate red/green (a*);
color coordinate yellow/blue (b*);
free amino nitrogen (PAN);
ammonia content;
urea content;
calcium content;
acidity;
rheological behavior;
strength;
degree of syneresis;
thermal behavior (DSC)

This list does not claim to be complete. Depending on the milk product used, products based primarily and mainly on milk, including cheeses or milk alternatives, it is generally possible to define further parameters. The informative value of the method according to the invention depends on the selection and number of parameters for which the products are tested. It has proven to be useful to select at least 5, preferably at least 7 parameters. The larger the scope of the set, the less important the informative value of an individual parameter.

Most parameters, such as the pH value, the dissolved amount of oxygen and the content of free amino nitrogen, urea, calcium and acids can be determined using the usual ana-lytic methods, which are notoriously known to the skilled person from food analysis and dairy science and which need not be presented in more detail here.

The instability index is determined using the LUMiSizer. This is a device that measures the particle and droplet velocity during creaming and sedimentation processes and determines the particle size distribution in accordance with DIN ISO 13318-2. A description of the device and the technology can be found here:
https://www.lum-gmbh.com/lumisizer_de.html#:˜:text=Der%20LUMiSizer%20ist%20das%20einzige,Systeme%20%E2%80%93%20Sie%20haben%20die%20Wahl

The brightness color value L′ and the two color coordinates a* and b* belong to the so-called L*a*b* color system, which is also known as the CIELAB system. It is the most commonly used system for color measurement today and is widely used in almost all areas of application. It was defined by the CIE in 1976 as one of the equidistant color spaces to counter the main problem of the original Yxy system: equal geometric difference distances in the x,y color triangle do not lead to perceived equal color differences. The color space of the L*a*b* system is characterized by the brightness L* and the color coordinates a* and b*. The signs indicate the color direction: +a* indicates a red component, −a* points in the direction of green. Accordingly, +b* stands for yellow and −b* for blue. At the origin of the coordinates (axis intersection point) is a neutral gray without any chromaticity. With increasing a*b* values, i.e., the further away the color location is from the center, the greater the chromaticity. The CR 400 or 410 device has proven to be suitable for determining the color values in the context of the method according to the invention. Further information can be found in the consumer information “Exact color communication” from Konica Minolta.

### Principal Component Analysis (PCA)

The data pool obtained in this way is evaluated in the next step with the help of the so-called “Principal Component Analysis” (PCA), which is also referred to as principal component analysis.

This is an established statistical method for analyzing large data sets with a high number of parameters per observation, for improving the interpretability of data while retaining a maximum amount of information and for visualizing multidimensional data. In formal terms, PCA is a statistical technique for reducing the dimensionality of a data set. This is done by a linear transformation of the data into a new coordinate system in which (most of) the variation in the data can be described with fewer dimensions than in the original data. In the method according to the invention, only the first two principal components are used to represent the data in two dimensions and to visually identify clusters of closely related data points.

In PCA data analysis, the first principal component of a set of p variables, which are assumed to be jointly normally distributed, becomes the derived variable, which is formed as a linear combination of the original variables and explains the largest variance. The second principal component explains the largest variance in what remains when the effect of the first component is removed, and we can continue with p iterations until all the variance is explained. PCA is most commonly used when many of the variables are highly correlated with each other and it is desirable to reduce their number to an independent set. Accordingly, the first or second principal component can be defined as a direction that maximizes the variance of the projected data. PCA is the simplest of the true eigenvector-based multivariate analyses and is closely related to factor analysis. A detailed description of the method can be found, for example, in https://en.wikipedia.org/wiki/Principal_component_analysis.

Briefly summarized, PCA provides a coordinate system in which the component with the largest variance or deviation (“first component”) is plotted against the component with the second largest variance (“second component”) for each data point. The more the data points are distributed over the coordinate field and the greater the distance between the data points of the aged samples and the data points of the fresh samples, the more pronounced the ageing effect is in total and the more meaningful the results of the method are in total. The PCA therefore serves the particular purpose of determining whether the selected test parameters are actually suitable for mapping the assumed change in the samples over time. If this is not the case, the set of test parameters must either be changed or supplemented.

### Partial Least Square (PLS) Calibration Method

Partial Least Squares Path Modeling or Partial Least Squares Structural Equation Modeling (PLS-PM, PLS-SEM) is a method for structural equation modeling that enables the estimation of complex cause-effect relationships in path models with latent variables.

It is a component-based estimation approach that differs from covariance-based structural equation modeling. In contrast to these structural equation modelling approaches, PLS-PM does not fit a joint factor model to the data, but a composite model, thus maximizing the amount of variance explained. In addition, PLS-PM is also able to consistently estimate certain parameters of common factor models through an adjustment called consistent PLS-PM (PLSc-PM). Another related development is factor-based PLS-PM (PLSF), a variant that uses PLSc-PM as the basis for estimating the factors in common factor models; this method significantly increases the number of common factor model parameters that can be estimated and effectively bridges the gap between classical PLS-PM and covariance-based structural equation modeling

The PLS-PM structural equation model is composed of two sub-models: the measurement models and the structural model. The measurement models represent the relationships between the observed data and the latent variables. The structural model represents the relationships between the latent variables.

An iterative algorithm solves the structural equation model by estimating the latent variables alternately using the measurement model and the structural model, hence the name of the method: partial. The measurement model estimates the latent variables as a weighted sum of the manifest variables. The structural model estimates the latent variables using simple or multiple linear regression between the latent variables estimated by the measurement model. This algorithm is repeated until convergence is achieved [see also Avkiram et. al, “Partial Least Squares Structural Equation Modeling”, Springer Verlag (2018)].

Briefly summarized, the PLS also provides a coordinate system based on the same data set, in which the component with the largest variance or deviation (“first component”) is plotted against the component with the second largest variance (“second component”) for each data point. At the same time, however, it calculates a value for an assessment criterion (“response”), for example the age of the sample, for each data point. This calculated value is then plotted in a calibration diagram against the actual value of the sample—for example, the known age. A regression line is then laid through the data cloud, which serves as a calibration line and can then be used to classify an unknown sample in terms of its age based on its physicochemical parameters.

### EXAMPLES

### Example 1A/B

### Selection of Parameters and Generation of Data

The process according to the invention for skimmed milk (fat content 0.3% by weight) is explained below. In the first step, the following parameters were selected from those available:
dissolved oxygen quantity
pH value
InI=instability index (Lumisizer)
brightness color value white/black (L*)
color coordinate red/green (a*)
color coordinate yellow/blue (b*)
free amino nitrogen (PAN)
ammonia content
urea content

A total of 4 skimmed milk samples in 0.2- or 1-liter format were taken; these constituted the first set of samples (P1 to P4). The samples were analyzed for the selected parameters (sample age: 0 weeks)

Each of these samples was then stored in the dark at 20° C. for a period of 22 weeks. At two-week intervals, new samples (P1.1, P2.1 . . . P1) were taken from each of the original samples and also analyzed according to the selected parameters. In this way, 11 aging stages were mapped for each of the original 4 samples. Thus, in the following table, sample 3 represents the 3rd sample, freshly bottled, while P3.9 represents the same sample in ageing stage 9, i.e., after 18 weeks of storage. The data set determined in this way is summarized in Table 1.

TABLE 1
Data set of quality measurement

| | | Age | O₂ diss. | | | | | | PAN | NH₄ | Urea |
|---|---|---|---|---|---|---|---|---|---|---|---|
| Sample | Format | [w] | [mg/l] | pH | Inl | L* | a* | b* | [mg/l] | [mg/l] | [mg/l] |
| 1 | 0.2 | 0 | 8 | 6.65 | 0.525 | 89.77 | −5.59 | 10.62 | 25 | 3 | 221 |
| 2 | 1 | 0 | 7.2 | 6.64 | 0.516 | 88.72 | −5.42 | 10.25 | 25 | 2 | 210 |
| 3 | 1 | 0 | 6.74 | 6.63 | 0.522 | 88.05 | −5.01 | 10.13 | 26 | 1 | 206 |
| 4 | 0.2 | 0 | 7.13 | 6.64 | 0.525 | 88.02 | −5.04 | 10 | 25 | 1 | 205 |
| 1.1 | 0.2 | 2 | 5.42 | 6.62 | 0.553 | 88.89 | −5.07 | 10.74 | 28 | 3 | 210 |
| 2.1 | 1 | 2 | 4.67 | 6.62 | 0.563 | 89.17 | −4.83 | 11.06 | 27 | 3 | 220 |
| 3.1 | 1 | 2 | 5.53 | 6.64 | 0.537 | 88.07 | −5.01 | 10.97 | 26 | 2 | 208 |
| 4.1 | 0.2 | 2 | 6.15 | 6.64 | 0.538 | 88.1 | −5.05 | 10.95 | 27 | 2 | 204 |
| 1.2 | 0.2 | 4 | 5.24 | 6.62 | 0.561 | 88.27 | −5.11 | 10.36 | 25 | 3 | 218 |
| 2.2 | 1 | 4 | 4.4 | 6.63 | 0.574 | 88.3 | −4.94 | 10.9 | 26 | 4 | 226 |
| 3.2 | 1 | 4 | 4.65 | 6.62 | 0.547 | 88.58 | −5.33 | 10.63 | 27 | 3 | 231 |
| 4.2 | 0.2 | 4 | 6.13 | 6.62 | 0.547 | 88.6 | −5.36 | 10.39 | 27 | 3 | 237 |
| 1.3 | 0.2 | 6 | 5.5 | 6.6 | 0.563 | 87.88 | −5.14 | 9.61 | 27 | 4 | 222 |
| 2.3 | 1 | 6 | 5.19 | 6.61 | 0.573 | 86.93 | −4.71 | 9.84 | 27 | 5 | 221 |
| 3.3 | 1 | 6 | 5.07 | 6.61 | 0.575 | 88.8 | −5.02 | 11 | 27 | 3 | 209 |
| 4.3 | 0.2 | 6 | 5.99 | 6.62 | 0.545 | 88.79 | −5.1 | 10.92 | 26 | 4 | 206 |
| 1.4 | 0.2 | 8 | 5.16 | 6.6 | 0.566 | 88.32 | −5.05 | 10.26 | 28 | 5 | 232 |
| 2.4 | 1 | 8 | 4.71 | 6.61 | 0.587 | 88.74 | −4.48 | 11 | 25 | 6 | 241 |
| 3.4 | 1 | 8 | 4.73 | 6.61 | 0.567 | 88.05 | −4.99 | 10.77 | 27 | 3 | 211 |
| 4.4 | 0.2 | 8 | 5.7 | 6.62 | 0.581 | 88.19 | −4.92 | 10.83 | 29 | 4 | 214 |
| 1.5 | 0.2 | 10 | 5.29 | 6.6 | 0.573 | 88.35 | −4.98 | 10.15 | 28 | 5 | 224 |
| 2.5 | 1 | 10 | 3.95 | 6.6 | 0.599 | 88.54 | −4.61 | 11.09 | 28 | 6 | 233 |
| 3.5 | 1 | 10 | 5.18 | 6.62 | 0.559 | 88.54 | −4.85 | 10.76 | 26 | 4 | 213 |
| 4.5 | 0.2 | 10 | 5.73 | 6.62 | 0.573 | 88.59 | −4.92 | 10.66 | 16 | 0 | 228 |
| 1.6 | 0.2 | 12 | 4.98 | 6.61 | 0.55 | 87.95 | −5.17 | 10.05 | 27 | 6 | 228 |
| 2.6 | 1 | 12 | 4.08 | 6.61 | 0.588 | 88.35 | −4.7 | 10.89 | 26 | 5 | 205 |
| 3.6 | 1 | 12 | 5.64 | 6.59 | 0.559 | 88.63 | −4.79 | 10.91 | 25 | 1 | 110 |
| 4.6 | 0.2 | 12 | 7.15 | 6.61 | 0.565 | 88.57 | −4.93 | 10.69 | 21 | 4 | 180 |
| 1.7 | 0.2 | 14 | 5.77 | 6.62 | 0.576 | 88.13 | −4.98 | 10.29 | 27 | 6 | 232 |
| 2.7 | 1 | 14 | 4.86 | 6.6 | 0.568 | 88.41 | −4.44 | 10.92 | 27 | 6 | 224 |
| 3.7 | 1 | 14 | 5.67 | 6.58 | 0.571 | 88.45 | −4.72 | 10.75 | 24 | 3 | 183 |
| 4.7 | 0.2 | 14 | 6.31 | 6.6 | 0.573 | 88.34 | −4.83 | 10.54 | 23 | 3 | 232 |
| 1.8 | 0.2 | 16 | 6.04 | 6.62 | 0.578 | 87.75 | −4.64 | 10.5 | 25 | 5 | 230 |
| 2.8 | 1 | 16 | 4.02 | 6.62 | 0.579 | 87.86 | −4.27 | 11.02 | 28 | 6 | 228 |
| 3.8 | 1 | 16 | 5.31 | 6.58 | 0.559 | 88.36 | −4.76 | 10.84 | 24 | 4 | 187 |
| 4.8 | 0.2 | 16 | 6.04 | 6.58 | 0.563 | 88.23 | −4.98 | 10.56 | 27 | 4 | 200 |
| 1.9 | 0.2 | 18 | 5.89 | 6.6 | 0.559 | 88.09 | −4.84 | 10.62 | 30 | 8 | 221 |
| 2.9 | 1 | 18 | 4.65 | 6.59 | 0.585 | 88.24 | −4.47 | 11.05 | 29 | 9 | 230 |
| 3.9 | 1 | 18 | 5.85 | 6.58 | 0.558 | 88.34 | −4.8 | 10.67 | 25 | 4 | 228 |
| 4.9 | 0.2 | 18 | 6.95 | 6.59 | 0.568 | 88.23 | −4.89 | 10.56 | 27 | 5 | 228 |
| 1.10 | 0.2 | 20 | 5.76 | 6.59 | 0.567 | 87.89 | −4.83 | 10.49 | 28 | 8 | 233 |
| 2.10 | 1 | 20 | 4.21 | 6.6 | 0.58 | 88.02 | −4.51 | 10.9 | 27 | 8 | 218 |
| 3.10 | 1 | 20 | 5.48 | 6.58 | 0.564 | 88.16 | −5.22 | 10.54 | 22 | 8 | 211 |
| 4.10 | 0.2 | 20 | 5.32 | 6.59 | 0.563 | 88.33 | −5.04 | 10.96 | 25 | 5 | 177 |
| 1.11 | 0.2 | 22 | 5.87 | 6.59 | 0.571 | 88.14 | −4.8 | 10.76 | 29 | 7 | 218 |
| 2.11 | 1 | 22 | 4.61 | 6.59 | 0.578 | 88.31 | −4.43 | 11.16 | 29 | 9 | 237 |
| 3.11 | 1 | 22 | 5.39 | 6.55 | 0.575 | 88.26 | −4.9 | 10.89 | 27 | 5 | 180 |
| 4.11 | 0.2 | 22 | 5.72 | 6.54 | 0.565 | 88.24 | −4.9 | 10.82 | 27 | 6 | 228 |

### Example 1C

### Checking the Correlation of the Data Using PCA=Principal Component Analysis

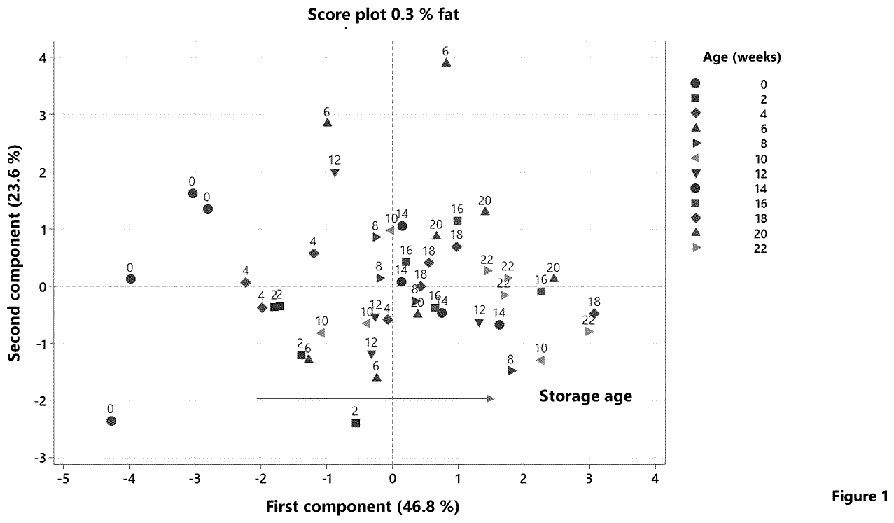

The third step of the method according to the invention is shown in FIG. 1. The previously obtained data set is processed using Principal Component Analysis. The PCA provides a coordinate system which is formed from the first and second principal components. In FIG. 1, the explained variance of principal component 1 is 46.8%. That of the second principal component is 23.6%. Overall, the PCA thus explains 70.4% of the variance in the original data. Knowing which parameters characterize the two principal components is irrelevant for the procedure, as this step is only concerned with bringing the variance to a scalable size.

As can be seen from the diagram, there is a large shift in relation to the original data set at t=0. This means that an ageing effect is actually present, which is examined in more detail in the fourth step below. If the data points were all in the immediate vicinity of the original t=0 values, this would conversely mean that there is no time-dependent effect; the procedure would therefore be aborted at this point. This step therefore serves to determine whether the method according to the invention can be applied to the products to be examined at all. If not, the parameters for collecting the data set must be supplemented or changed.

### Example 1D

### Setting Up the PLS=Partial Least Squares Calibration Model

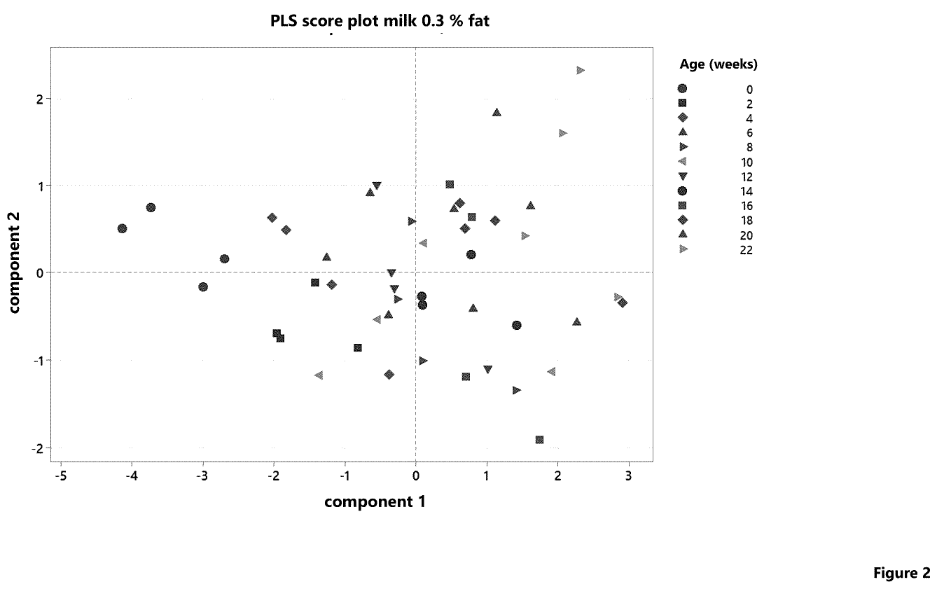

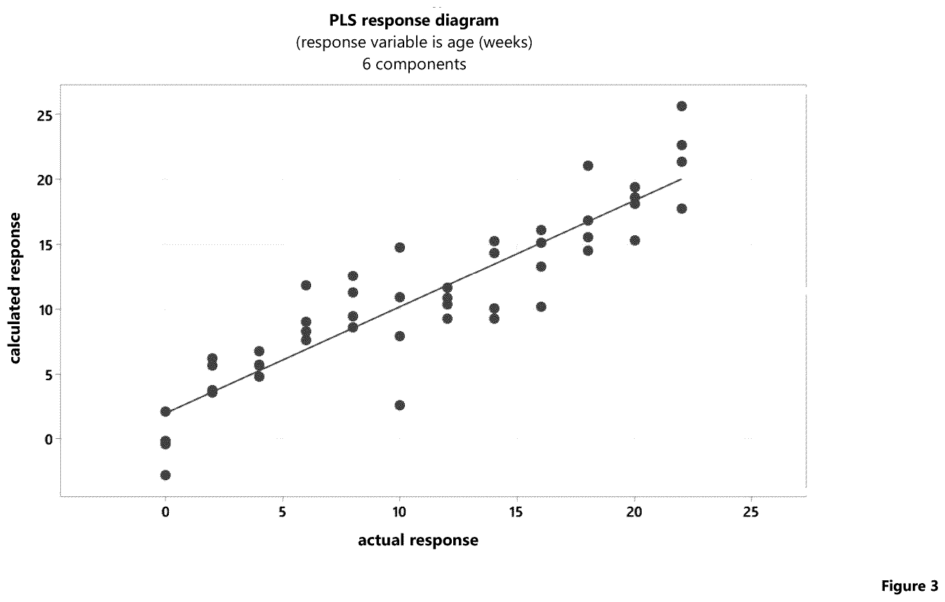

The fourth step of the method according to the invention is shown in FIGS. 2 and 3. After confirming in the previous step that the set of selected test parameters leads to a data set that actually reflects a time-dependent change in the samples, the same data set is prepared this time using the partial least square calibration method. This again provides a diagram in which the largest and second largest variance span the coordinate system (FIG. 2), but also provides a calculated measured value for a given parameter (“response”)—in this case the age of the sample. If the calculated measured values, i.e. the calculated age of the sample, are plotted against the actual and known age of this sample, a calibration curve can be laid out through the point array (FIG. 3). The calibration curve can then be used to determine the age of an undated sample, for example.

What claimed is:

1. A method of assessing the stability and quality of milk products, primary or main milk-based products including cheese and plant-based milk alternatives over storage time, comprising or consisting of the following steps:
(a) providing a first set of samples (P1.0, P2.0 . . . Pn.0) of a dairy product, a primary or main milk-based product including cheese, or a plant-based dairy alternative;
(b) ageing the samples from step (a) over defined periods of time to obtain an nth set of samples (P1.1, P1.2 . . . P2.1, P2.2 . . . Pn.x);
(c) creating a set of test parameters;
(d) applying the specified test parameters to each of the individual samples of the first and nth sample set, generating a data set;
(e) checking the correlation of the data in the data set using Principal Component Analysis (PCA) to obtain a coordinate system (score plot) and determining the largest variance of the data as the first component and the second largest variance of the data as the second component;
(f) Performing a Partial Least Square Analysis (PLS) on the data;
(g) generating a partial least square (PLS) calibration model to obtain a PLS response plot (calibration line), wherein the calculated response for each data point is plotted against the corresponding measured response of each data point; and
(h) evaluating an external sample with respect to the specified evaluation criterion using the calibration grades.

2. The method according to claim 1, characterized in that the milk products or plant-based milk alternatives are selected from the group formed by whole milk, skimmed milk, UHT milk, milk powders, cream, whey, curd, cheese, yoghurt or their plant-based alternatives.

3. The method according to claims 1 and/or 2, characterized in that the first set of samples comprises 3 to 10 samples.

4. The method according to at least one of claims 1 to 3, characterized in that the second set of samples comprises the samples of the first set of samples, each of which has been stored for a period of 1 to 25 weeks.

5. The method according to at least one of claims 1 to 4, characterized in that the samples have a volume of between 10 ml and 100 L.

6. The method according to at least one of claims 1 to 5, characterized in that the set of test parameters comprises at least two of the following parameters:
dissolved oxygen quantity;
pH value;
instability index (Lumisizer);
Brightness color value white/black (L*);
color coordinate red/green (a*);
color coordinate yellow/blue (b*);
free amino nitrogen (PAN);
ammonia content;
urea content;
calcium content;
acidity;
rheological behavior;
strength;
degree of syneresis; and
thermal behavior (DSC).

7. The method according to claim 6, characterized in that the set of test parameters contains at least 5, preferably at least 7 parameters.

8. The method according to at least one of claims 1 to 7, characterized in that the age of the sample is selected as the assessment criterion to be applied.
